(19) United States
(12) Patent Application Publication
(10) Pub. No.: US 2025/0290931 A1
(43) Pub. Date: Sep. 18, 2025

(54) METHODS FOR PROTEIN-SDS COMPLEX DECOUPLING FOR PROTEIN MASS SPECTROMETRY ANALYSIS

(71) Applicant: GMJ TECHNOLOGIES, INC., Seattle, WA (US)

(72) Inventor: Oluwatosin O. Dada, Everett, WA (US)

(21) Appl. No.: 19/076,283

(22) Filed: Mar. 11, 2025

Related U.S. Application Data
(60) Provisional application No. 63/564,871, filed on Mar. 13, 2024.

(51) Int. Cl. G01N 33/68; G01N 27/447

(52) U.S. Cl. CPC G01N 33/6848; G01N 27/44721; G01N 27/44747; G01N 27/44791; G01N 2560/00

(57) ABSTRACT

Decoupling of protein-SDS coupling through a use of an amphipathic co-solvent such as MPD in combination with a co-reagent such as an organic co-solvent allows to decouple protein-SDS complexes and make the proteins suitable for analysis by mass spectrometry. The decoupling can be done offline, with the protein-SDS complexes being decoupled prior to being loaded onto an apparatus such as LC-MS or CE-MS apparatus. Alternatively, the decoupling can be done inline to minimize the time the protein is exposed to the decoupling reagents, with the decoupling reagents being added to a capillary of a CE-MS apparatus into which the sample of the protein-SDS complex has previously been injected.

This invention was made with government support under Grant No. 1R43GM151899 awarded by the National Institutes of Health. The government has certain rights in the invention.

### FIELD

The invention relates in general to protein analysis, and in particular, to methods for protein-SDS complex decoupling for protein mass spectrometry analysis.

### BACKGROUND

Sodium dodecyl sulfate (SDS) is commonly used in protein bioanalysis. In particular, one of the most common uses of the reagent is during SDS-PAGE gel electrophoresis, where SDS binds the proteins being analyzed to denature them and impart to them a negative charge that results in protein length being the primary determinant of how quickly the protein-SDS complexes moves during the electrophoresis. While useful for this particular purpose, the presence of SDS interferes with another key protein analysis technique: mass spectrometry.

Mass spectrometry is an important tool for characterizing protein mass and allows for protein identification and protein complex elucidation, three-dimensional protein structure determination, protein sequencing, determining presence of post-translational modifications on the proteins being analyzed, and proteome analysis. Other applications of mass spectrometry in protein bioanalysis are abundant. While different variations of mass spectrometry exist, the electrospray ionization (ESI) mass spectrometry has been particularly useful in the study of proteins due to being a soft ionization technique that generally does not cause protein fragmentation upon ionization and often does not disrupt non-covalent protein interactions.

However, when a protein of interest is in a complex with SDS, such as after being separated from other proteins through SDS-PAGE gel electrophoresis, SDS interferes with and suppresses protein mass spectrometry signal. In particular, SDS ionizes more efficiently than proteins and even a small amount of SDS in the protein sample being analyzed can overwhelm the mass spectrometry analyzer. As a result, currently, before a protein that is in complex with SDS can undergo mass spectrometry, the protein is subjected to multiple precipitation and reconstitution steps to separate the protein from SDS. Such precipitation and reconstitution steps are time consuming, thus limiting the usefulness of mass spectrometry, especially for ESI mass spectrometry. Further, such precipitation and reconstitution steps can decrease protein yield and subject the protein to additional opportunities for degradation.

Attempts have been made to interfere with the effects of SDS on proteins. In particular, “Michaux, C., Pomroy, N. C., & Privé, G. G. (2008). Refolding SDS-denatured proteins by the addition of amphipathic cosolvents. Journal of molecular biology, 375 (5), 1477-1488,” and “Michaux, C., Pouyez, J., Wouters, J., & Privé, G. G. (2008). Protecting role of cosolvents in protein denaturation by SDS: a structural study. BMC structural biology, 8, 1-7,” describe that 2-methyl-2,4-pentanediol (MPD) can protect both structure and function of proteins from effects of SDS, and in some cases, can promote refolding of the protein denatured by SDS. However, the addition of MPD has empirically been shown to be insufficient to cause a decoupling of already created protein-SDS complexes to a degree that makes protein analysis by mass spectrometry practicable.

Accordingly, there is a need for a way to quickly and efficiently separate proteins from SDS in protein-SDS complexes for mass spectrometry analysis while minimizing protein loss.

### SUMMARY

Decoupling of protein-SDS coupling through a use of an amphipathic co-solvent such as MPD in combination with a co-reagent such as an organic co-solvent (with or without a protein stabilizer) allows to decouple protein-SDS complexes and make the proteins suitable for analysis by mass spectrometry. The decoupling can be done offline, with the protein-SDS complexes being decoupled prior to being loaded onto an apparatus such as LC-MS or CE-MS apparatus. Alternatively, the decoupling can be done inline to minimize the time the protein is exposed to the decoupling reagents, with the decoupling reagents being added to a capillary of a CE-MS apparatus into which the sample of the protein-SDS complex has previously been injected.

In one embodiment, a method for protein-SDS complex decoupling is provided. The method includes obtaining a buffer including molecules of a protein and molecules of sodium dodecyl sulfate (SDS), wherein at least some of the protein molecules form complexes with the SDS molecules; and decoupling at least some of the protein-SDS complexes by adding an amphipathic co-solvent and a co-reagent to the buffer.

In one embodiment, a method for offline protein-SDS complex decoupling for protein mass spectrometry analysis is provided. A sample of a protein included in a buffer solution that includes sodium dodecyl sulfate (SDS) is obtained. An amphipathic co-solvent is added to the buffer solution. A co-reagent is added to the buffer solution after the addition of the amphipathic co-solvent, wherein the additions of the amphipathic organic co-solvent and the co-reagent cause a decoupling of the SDS from at least a portion of the protein in the sample. The decoupled protein is analyzed using mass spectrometry.

In a further embodiment, a method for inline protein-SDS complex decoupling for capillary electrophoresis-mass spectrometry analysis is provided. A sample of a protein included in a buffer solution that includes sodium dodecyl sulfate (SDS) is obtained. Decoupling reagents that include an amphipathic co-solvent and a co-reagent are prepared. A capillary of a capillary electrophoresis-mass spectrometry (CE-MS) system is loaded with a background electrolyte. The protein sample is injected into the capillary. The de-coupling reagents are injected into the capillary, wherein the injection of the de-coupling reagents causes a decoupling of the SDS from at least a portion of the protein in the sample. Voltage in a positive polarity is applied to the capillary to start separation of the decoupled protein from the SDS via capillary electrophoresis, wherein the separated protein is analyzed via a mass spectrometer of the CE-MS system.

Still other embodiments of the present invention will become readily apparent to those skilled in the art from the following detailed description, wherein is described embodiments of the invention by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF THE DRAWINGS

FIG. 1 is a diagram illustrating formation of a protein-SDS complex and decoupling of the protein-SDS complex through addition of an amphipathic co-solvent and a co-reagent in accordance with one embodiment.

FIG. 2 is a flow diagram showing a method for protein-SDS complex decoupling for protein mass spectrometry analysis in accordance with one embodiment.

FIG. 3 is a flow diagram showing a method for offline protein-SDS complex decoupling for protein mass spectrometry analysis for use in the method of FIG. 2 in accordance with one embodiment.

FIG. 4 is a flow diagram showing a method for inline protein-SDS complex decoupling for capillary electrophoresis-mass spectrometry analysis for use in the method of FIG. 2 in accordance with one embodiment.

FIGS. 5A-5B are capillary electrophoresis profiles illustrating effectiveness of use of the methods of FIGS. 2 and 3.

FIGS. 6A-6B are capillary electrophoresis profiles illustrating effectiveness of use of the methods of FIGS. 3 and 4.

FIG. 7 is a diagram illustrating performance of inline separation of proteins from SDS in a capillary of a CE-MS apparatus.

### DETAILED DESCRIPTION

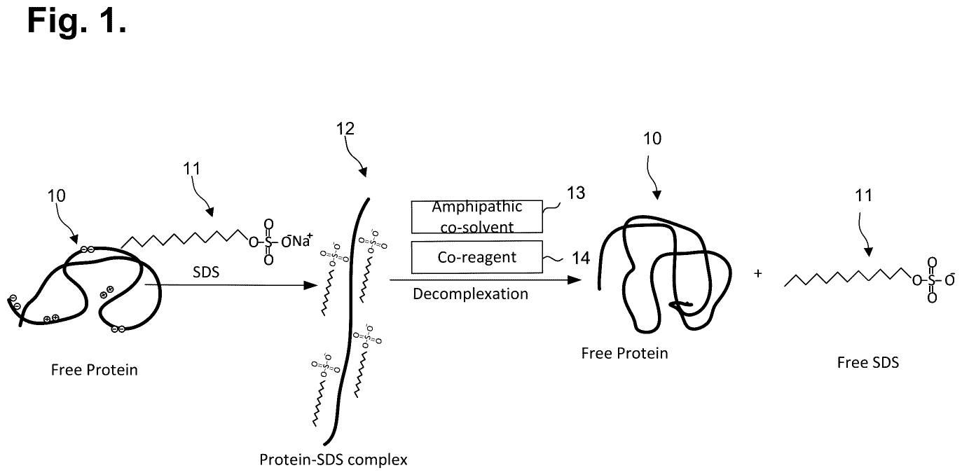

Use of an amphipathic co-solvent such as MPD in combination with a co-reagent such as an organic co-solvent allows to decouple protein-SDS complexes and make the proteins suitable for analysis by mass spectrometry. FIG. 1 is a diagram illustrating formation of a protein-SDS complex 12 and decoupling of the protein-SDS complex 12 through addition of an amphipathic co-solvent 13 and a co-reagent 14 in accordance with one embodiment. As mentioned above, binding of SDS 11 to a protein 10 denatures the protein and imparts a negative charge to the protein-SDS complex 12. The addition of the amphipathic co-solvent 13 in combination with a co-reagent 14 separates the protein-SDS complex 12 back into constituent parts 10, 11. In addition to the amphipathic co-solvent 13 and the co-reagent 14, a protein stabilizer such as one or more osmolytes or urea (though other protein stabilizers can also be used) can also optionally be added to the protein-SDS 12 complex buffer to prevent the decoupled protein 10 from precipitating from solution. The conformation of the protein 10 freed from the protein-SDS complex 12 may not be the same as before the protein 10 formed the complex with SDS 11. The amphipathic co-solvent 13 can include MPD or one or more analogs of MPD, such as 2-ethyl-2,4-pentadiol 2,4-dimethyl-2,4-pentanediol, and hexanediol, though other kinds of amphipathic co-solvents 13 are also possible. The co-reagent 14 can include an organic co-solvent, such as acetonitrile or isopropanol, though other organic co-solvents are also possible. Other types of co-reagents are also possible. For example, the organic co-solvent can include methanol, dimethyl sulfoxide (DMSO), dimethyl sulfide (DMS), dimethylacetamide (DMA), dimethylformamide (DMF), and salts such as potassium chloride and phosphate salts. Still other types of co-reagents are possible.

Once a protein-SDS 12 complex is decoupled into constituent parts, the resulting protein 10 and SDS 11 are subjected to a separation technique, such as liquid chromatography or capillary electrophoresis, prior to being analyzed by mass spectrometry. The de-coupling can be performed before the proteins-SDS complex is loaded onto the separation system (“offline”). Alternatively, to minimize the time that the protein is exposed to the amphipathic co-solvent 13 and the co-reagent 14 and minimize any potential damage to the protein from these decoupling reagents, the de-coupling can be performed when the protein-SDS complex 12 is already loaded onto a capillary of the capillary electrophoresis-mass spectrometry (“CE-MS”) system (“inline”). The decoupling methods, including the offline and inline separation methods, are described below.

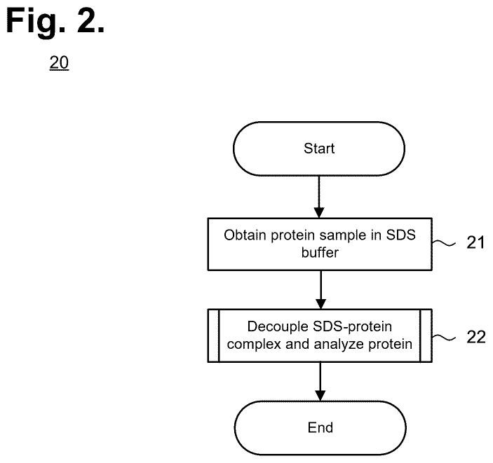

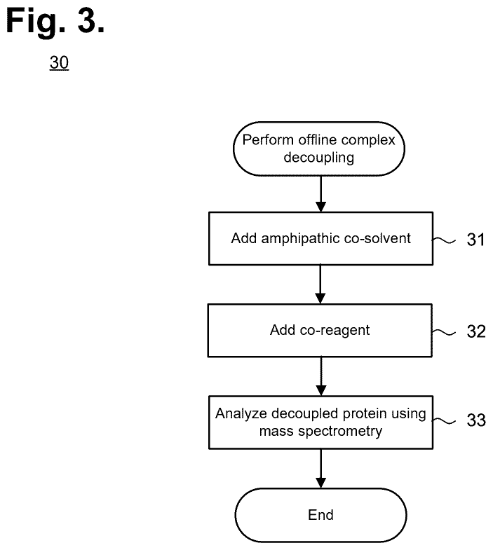

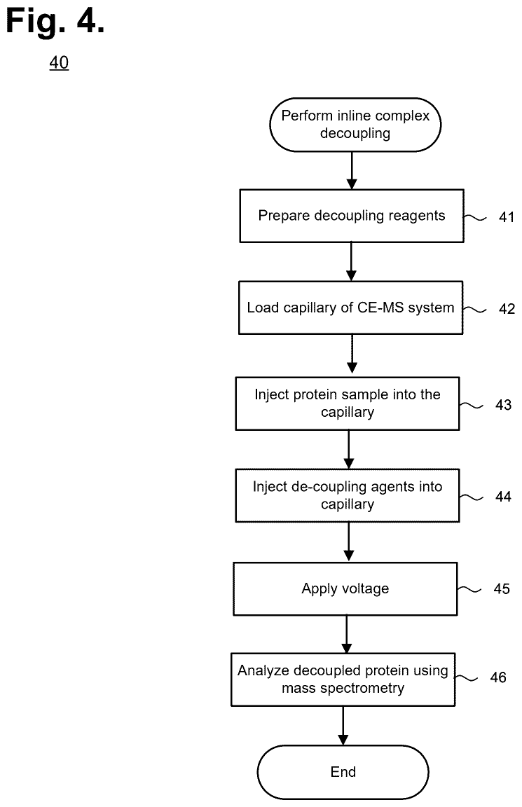

FIG. 2 is a flow diagram showing a method 20 for protein-SDS complex decoupling for protein mass spectrometry analysis in accordance with one embodiment. A sample of a protein 10 in a buffer solution that includes SDS 11 (and thus at least a portion of the protein 10 molecules being in a complex 12 with SDS 11 molecules) is obtained (step 21). A decoupling of at least some of the complexes 12 into constituent protein 10 and SDS 11 molecules is caused through addition of an amphipathic co-solvent and a co-reagent 14 and the decoupled protein 10 molecules are analyzed through mass spectrometry (step 22), as further described below with reference to FIGS. 3 and 4, ending the method 20. The decoupling (step 22) can be performed offline, as further described with reference to FIG. 3. Alternatively, the decoupling (step 22) can be performed inline, as further described below with reference to FIG. 4.

FIG. 3 is a flow diagram showing a method 30 for offline protein-SDS complex decoupling for protein mass spectrometry analysis for use in the method 20 of FIG. 2 in accordance with one embodiment. An amphipathic co-solvent 13 is added to the buffer solution (step 31). In one embodiment, the volume of the amphipathic co-solvent 13 added to the buffer can be between up 0.1% to 50% of the volume of the buffer (depending on the concentration of the protein-SDS complex 13 and the properties of the protein 10 in the complex 12 (such as the protein's size and structure, though other properties are also possible), though in a further embodiment, other ratios of the volume of the amphipathic co-solvent to the volume of the buffer are possible. The amphipathic co-solvent 13, when added to the buffer, can be pure or nearly pure (such as 99% purity). Alternatively, the amphipathic co-solvent 13 can be pre-mixed with the same buffer as the protein-SDS complex 12 is in, or pre-mixed with a protein stabilizer, such as one or more osmolytes or urea (though other protein stabilizers can also be used). The amphipathic co-solvent 13 can also be pre-mixed with the co-reagent 14 as described below.

Following the addition of the amphipathic co-solvent (step 31), the co-reagent 14 is added to the buffer (step 32). Steps 31 and 32 cannot be switched as the addition of the co-reagent 14 to the buffer before the addition of the amphipathic co-solvent 13 can cause the protein 10 (even as part of the protein-SDS complex 12) to precipitate from the buffer. However, steps 31 and 32 can be combined, with the amphipathic co-solvent 13 and the co-reagent 14 being pre-mixed and thus added at the same time. In one embodiment, the volume of the co-reagent 14 added to the buffer can be between up 0.1% to 20% of the volume of the buffer (depending on the concentration of the protein-SDS complex 12 and the properties of the protein 10 in the complex 12), though in a further embodiment, other ratios of the volume of the co-reagent 14 to the volume of the buffer are possible. The co-reagent 14, when added to the buffer can be pure or nearly pure (such as 99% purity). Alternatively, the co-reagent 13 can be pre-mixed with the same buffer as the protein-SDS complex 12 is in, or pre-mixed with a protein stabilizer, such as one or more osmolytes or urea (though other protein stabilizers can also be used). The co-reagent 14 can also be pre-mixed with the amphipathic co-solvent 13 as described above. The addition of both the amphipathic co-solvent 13 and the co-reagent 14 will cause the protein-SDS complexes 12 that came into contact with the decoupling reagents 13, 14 to disassociate, and the protein 10 released from the complex is analyzed using mass spectrometry (step 33), ending the method 20. The analysis in step 33 includes use of a separation technique coupled with mass spectrometry to physically separate SDS 11 molecules in the buffer from the protein molecules in the buffer 11 before they are analyzed through mass spectrometry. The mass spectrometry technique applied in step 24 can be ESI mass spectrometry, though in a further embodiment, other kinds of mass spectrometry can also be used. Thus, the mixture in step 33 could be subjected to liquid chromatography-mass spectrometry (LC-MS) or CE-MS, allowing for both physical separation of the protein 10 from SDS 11 and mass spectrometry analysis of the protein 10 using the same system.

Inline decoupling reduces the time that the protein is exposed to the decoupling reagents (amphipathic co-solvent 13 and the co-reagent 14) when compared to offline decoupling and thus can be preferable to offline decoupling when involving proteins 10 that can be damaged by these decoupling reagents. FIG. 4 is a flow diagram showing a method 40 for inline protein-SDS complex 12 decoupling for capillary electrophoresis-mass spectrometry analysis for use in the method 20 of FIG. 2 in accordance with one embodiment. Decoupling reagents, including an amphipathic co-solvent 13 and a co-reagent 14 are prepared (step 41). A capillary of a CE-MS system is loaded with a background electrolyte (step 42). In one embodiment, the background electrolyte can be acetic acid, though in a further embodiment, other background electrolytes are also possible. For example, background electrolytes can include ammonium acetate, ammonium formate, tris(hydroxymethyl)aminomethane (“Tris”) buffer, ammonium bicarbonate, formic acid, though still other types of background electrolytes are also possible.

The protein sample is injected into the capillary loaded with the background electrolyte (step 43). The de-coupling reagents 13, 14 are injected into the capillary into which the protein sample has been injected (step 44). The ratios of the volumes of the amphipathic co-solvent 13 and the co-reagent 14 (and their purity) to the volume of the sample of the protein 10 can be the same as described above with reference to FIG. 3, though in a further embodiment, other ratios of the volumes and other purity of the decoupling reagents 13, 14 are possible. In one embodiment, the amphipathic co-solvent 13 is injected before the injection of the co-reagent 14 into the capillary; in a further embodiment, the amphipathic co-solvent 13 and the co-reagent 14 could be mixed together before the injection and injected into the capillary at the same time. In addition, a protein stabilizer such as one or more osmolytes or urea (though other protein stabilizers can also be used) could be premixed with the co-reagent 14, the amphipathic co-solvent 13, or both. The injection of the decoupling reagents 13, 14 causes a decoupling of the protein-SDS 12 complexes in the capillary that came into contact with the reagents 13, 14. Voltage is applied to the capillary in a positive polarity perform separation of the decoupled protein 10 from the SDS 11 via capillary electrophoresis (step 45). The separated protein is provided into the mass spectrometry portion of the CE-MS system and is analyzed by mass spectrometry (step 46), thus ending the method 30. The mass spectrometry technique applied in step 37 can be ESI mass spectrometry, though in a further embodiment, other kinds of mass spectrometry can also be used. The CE separation technique may be in multi-dimensional configuration where different modes of CE are combined to achieve higher resolution.

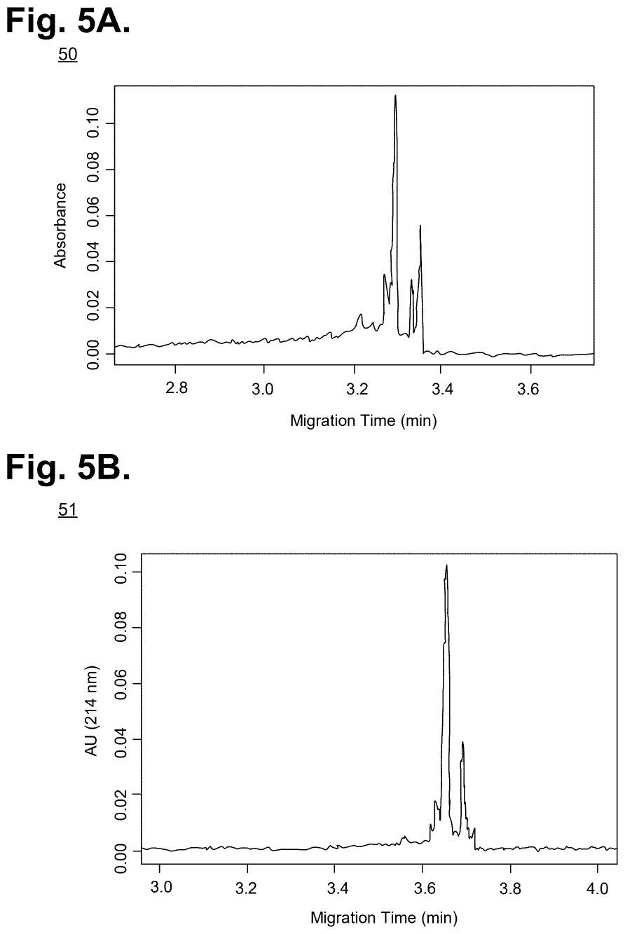

The techniques described above with reference to FIGS. 1-4 have been empirically validated. FIGS. 5A-5B are capillary electrophoresis profiles 50, 51 illustrating effectiveness of use of the method 20 of FIG. 2. FIG. 5A is a capillary electrophoresis profile 40 achieved by running 1 mg/ml Lysozyme protein that is in 50 mM Tris/10 mM Phosphate buffer (with no SDS 11 being present) through capillary electrophoresis. The background electrolyte used in the capillary electrophoresis was 5% acetic acid. The separation voltage applied to the capillary was +20 kV and the length of the capillary was 18 cm. The x-axis of the profile 50 is migration time and the y-axis is absorbance. The profile 50 of FIG. 5A serves as a control for the profile 51 of FIG. 5B. The profile 51 of FIG. 5B was obtained using the method 20 of FIG. 2 and the method 30 of FIG. 3. In particular, the profile 51 of FIG. 5B was achieved by obtaining 1 mg/mL Lysozyme in 50 mM Tris/0.25% SDS buffer; incubating the Lysozyme protein at 70° C. for 10 minutes to maximize the formation of Lysozyme-SDS complexes 12; adding 10% by volume of MPD (percentage determined by comparing to volume of added MPD to volume of the buffer) followed by adding 10% by volume of acetonitrile (percentage determined by comparing to volume of added acetonitrile to volume of the buffer). The buffer following the addition of acetonitrile was analyzed by capillary electrophoresis to produce the profile 51. The background electrolyte used during the capillary electrophoresis was 5% acetic acid and the separation voltage applied during the capillary electrophoresis was +20 kV. The length of the capillary was 18 cm. The x-axis of the profile 51 is migration time and the y-axis is absorbance.

The profile 51 of FIG. 4B shows that the addition of MPD and acetonitrile has disrupted the lysozyme-SDS complex. A protein 10 that is still bound to SDS 11 would not migrate in the same direction as a protein 10 that is free from SDS due to the charge difference. Accordingly, the dominant peak in both the profiles 50 and 51 being of approximately the same height and shape and being in the same location along the x-axis points to the lysozyme being completely decoupled from SDS in the profile 51 of FIG. 5B through the use of the method 20 of FIG. 2 and method 30 of FIG. 3.

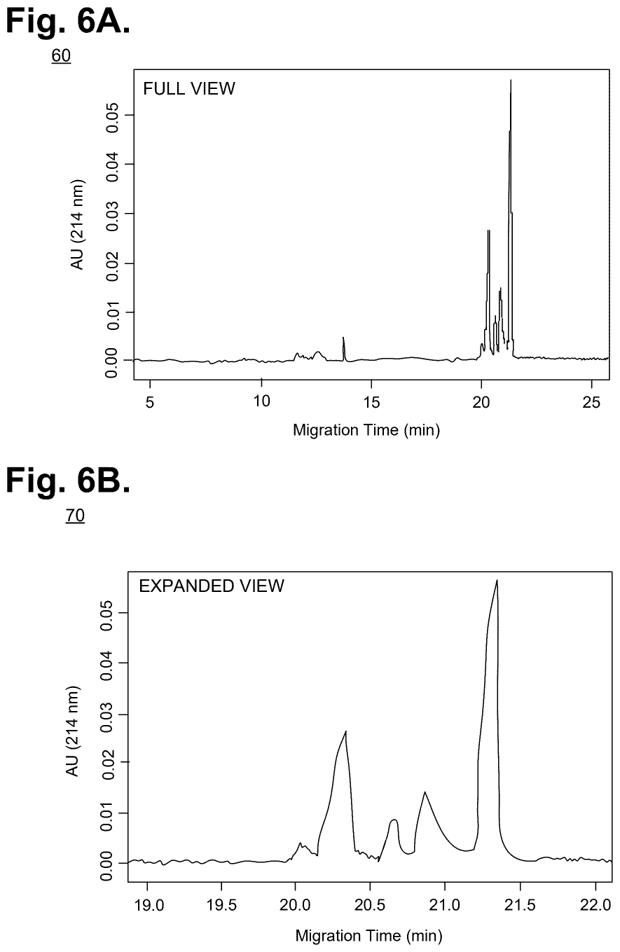

The inline protein-SDS complex 12 decoupling of the method 40 of FIG. 4 has also been empirically validated. FIGS. 6A-6B are capillary electrophoresis profiles 60, 70 illustrating effectiveness of use of the method 40 of FIG. 4. FIG. 6A is a capillary electrophoresis profile 60 achieved by running through capillary electrophoresis the following protein sample: 1 mg/mL Lysozyme, 0.5 mg/mL Myoglobin, 1 mg/mL Bovine Serum Albumin (BSA) in 50 mM Tris buffer, with pH of the buffer being, 8.8, and SDS being 0.25% (volume/volume) of the buffer. 5% acetic acid was used as the background electrolyte. The separation voltage was +20 kV. Initially, a two second plug of the sample was injected into the capillary, followed by an injection into the capillary of a two second plug of 50 mM Tris/20% MPD/15% acetonitrile (with the percentages being given by volume compared to the total volume of the mixture of Tris, MPD, and acetonitrile). As can be seen with reference to FIG. 6A, the inline decoupling was successful, with the proteins presenting well defined peaks during the CE-MS analysis (with the lysozyme presenting the first three peaks on the left, followed by a peak from BSA, followed by a peak from myoglobin). The x-axis of the profile 60 is migration time and the y-axis is absorbance. FIG. 6B shows the profile 70 that is an expanded view of the peaks of the profile 60 of FIG. 6A. The profiles 60, 70 of FIGS. 6A-6B show that the method 40 works for proteins of different sizes.

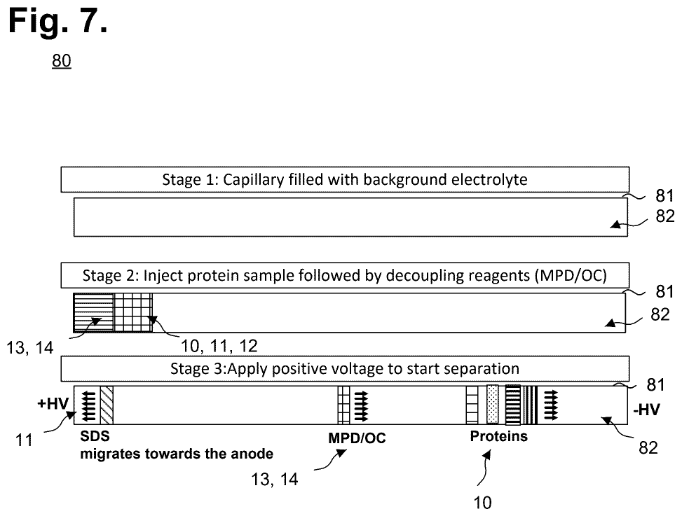

The inline separation of proteins 10 from SDS 11 seen with reference to FIGS. 6A-6B can also be illustrated with respect to the capillary used in the capillary electrophoresis. FIG. 7 is a diagram 80 illustrating performance of inline separation of proteins 10 from SDS 11 in a capillary 81 of a CE-MS apparatus. In stage 1, the capillary 81 is filled with a background electrolyte 82. In stage 2, a sample that includes protein-SDS complexes 12 is injected into the capillary 81 followed by an injection of the decoupling reagents 13, 14 (in the embodiment shown with reference to FIG. 7, MPD together with an organic co-solvent). As the protein-SDS complex 12 is exposed within the capillary 81 to the decoupling reagents 13, 14, the protein-SDS complexes 12 disassociate into the separate proteins 10 and SDS 11. Separation voltage is applied, forcing the SDS 11 to migrate towards the anode while the proteins 10 migrate towards the cathode, resulting in the separation of the protein 10 molecules from SDS 11 molecules and making the proteins 10 suitable for analysis by mass spectrometry.

In a further embodiment, the offline and inline decoupling techniques using an amphipathic co-solvent and a co-reagent described above could be applied to molecules other than proteins. For example, the techniques described above could be applied to decoupling lipids, nucleic acids, and organic molecules from SDS (or other similar molecules).

Prior to performing the methods described above with reference to FIGS. 2-4, SDS-Protein complexation is typically performed at an elevated temperature (70° C. or more) to achieve a complete SDS-protein binding. The effectiveness of MPD in decoupling SDS-protein complexes can vary depending on the efficiency of protein-SDS binding. MPD may partially decouple SDS-protein, reducing a number of the copies (molecules) of SDS on the protein significantly so that the protein can be analyzed by MS without SDS interference. The protein with a significantly smaller number of SDS copies can be analyzed by inline MS techniques such as LC-MS or CE-MS. However, temperature can also be utilized to enhance the effectiveness of the amphipathic co-solvent (such as MPD) decoupling activity. When temperature becomes a critical factor in enhancing MPD effectiveness, in the methods described above with reference to FIGS. 2-4, the amphipathic co-solvent is first added to the sample, incubated at 40° C. or higher (to a permissible level that maintains protein integrity) for 10 minutes or more. After incubation, an organic co-solvent is added, and the mixture is allowed to equilibrate for 10 minutes or more. Other co-reagents such as inorganic additives, protein stabilizers (osmolytes), urea, and more could be utilized to enhance MPD (or another amphipathic co-solvent) decoupling activities. In the method 30 described above with reference to FIG. 3, the protein may also be recovered after treating with amphipathic co-solvent, using varieties of approaches such as precipitation-resuspension process and affinity capturing.

While in the description above, the decoupled protein 10 is described as being analyzed via certain mass spectrometry techniques, in a further embodiment, other kinds of analytical techniques, including other mass spectrometry techniques, can be applied to the decoupled protein.

While the invention has been particularly shown and described as referenced to the embodiments thereof, those skilled in the art will understand that the foregoing and other changes in form and detail may be made therein without departing from the spirit and scope of the invention.

1. A method for protein-SDS complex decoupling, comprising:
obtaining a buffer comprising molecules of a protein and molecules of sodium dodecyl sulfate (SDS), wherein at least some of the protein molecules form complexes with the SDS molecules; and
decoupling at least some of the protein-SDS complexes by adding an amphipathic co-solvent, a protein stabilizer different than the amphipathic co-solvent, and a co-reagent to the buffer.

2. A method according to claim 1, wherein the co-reagent is added to the buffer after the addition of the amphipathic co-solvent, and wherein the buffer is incubated with the amphipathic co-solvent at at least 40° C. for at least 10 minutes prior to the addition of the co-reagent.

3. A method according to claim 1, wherein the amphipathic co-solvent comprises at least one of 2-Methyl-2,4-pentanediol (MPD) and one or more analogs of MPD.

4. A method according to claim 1, wherein the co-reagent comprises an organic co-solvent.

5. A method according to claim 1, wherein the organic co-solvent comprises one or more of acetonitrile and isopropanol.

6. A method according to claim 1, further comprising:
analyzing at least some of the protein molecules decoupled from the complexes.

7. A method according to claim 6, wherein the analysis comprises mass spectrometry.

8. A method according to claim 6, wherein the analysis comprises at least one of liquid chromatography-mass spectrometry and capillary electrophoresis-mass spectrometry.

9. A method according to claim 1, further comprising:
preparing the amphipathic co-solvent and the co-reagent;
loading a capillary of a capillary electrophoresis-mass spectrometry (CE-MS) system with a background electrolyte;
injecting the buffer into the capillary, wherein at least one of the amphipathic co-solvent and the co-reagent are pre-mixed with a protein stabilizer different than the amphipathic co-solvent, and wherein the addition of the amphipathic co-solvent and the co-reagent comprises injecting the amphipathic co-solvent and the co-reagent into the capillary; and
applying voltage in a positive or negative polarity to the capillary to start separation of the decoupled protein molecules from the SDS via capillary electrophoresis, wherein the separated protein molecules are analyzed via a mass spectrometer of the CE-MS system.

10. A method for offline protein-SDS complex decoupling for protein mass spectrometry analysis, comprising:
obtaining a sample of a protein comprised in a buffer solution comprising sodium dodecyl sulfate (SDS);
adding an amphipathic co-solvent to the buffer solution;
adding a co-reagent to the buffer solution after the addition of the amphipathic co-solvent, wherein at least one of the amphipathic co-solvent and the co-reagent are pre-mixed with a protein stabilizer different than the amphipathic co-solvent, and wherein the additions of the amphipathic organic co-solvent and the co-reagent cause a decoupling of the SDS from at least a portion of the protein in the sample; and
analyzing the decoupled protein using mass spectrometry.

11. A method according to claim 10, wherein the amphipathic co-solvent comprises at least one of 2-Methyl-2,4-pentanediol (MPD) and one or more analogs of MPD.

12. A method according to claim 10, wherein the co-reagent comprises an organic co-solvent.

13. A method according to claim 10, wherein the buffer is incubated with the amphipathic co-solvent at at least 40° C. for at least 10 minutes prior to the addition of the co-reagent.

14. A method according to claim 10, wherein the mass spectrometry is at least one of liquid chromatography-mass spectrometry and capillary electrophoresis-mass spectrometry.

15. A method for inline protein-SDS complex decoupling for capillary electrophoresis-mass spectrometry analysis, comprising:
obtaining a sample of a protein comprised in a buffer solution comprising sodium dodecyl sulfate (SDS);
preparing decoupling reagents comprising an amphipathic co-solvent, a protein stabilizer different than the amphipathic co-solvent, and a co-reagent;
loading a capillary of a capillary electrophoresis-mass spectrometry (CE-MS) system with a background electrolyte;
injecting the protein sample into the capillary;
injecting the de-coupling reagents into the capillary, wherein the injection of the de-coupling reagents causes a decoupling of the SDS from at least a portion of the protein in the sample; and
applying voltage in a positive or negative polarity to the capillary to start separation of the decoupled protein from the SDS via capillary electrophoresis, wherein the separated protein is analyzed via a mass spectrometer of the CE-MS system.

16. A method according to claim 15, wherein the amphipathic co-solvent comprises at least one of 2-Methyl-2,4-pentanediol (MPD) and one or more analogs of MPD.

17. A method according to claim 15, wherein the co-reagent comprises an organic co-solvent.

18. A method according to claim 17, wherein the organic co-solvent comprises one or more of acetonitrile and isopropanol.

19. A method according to claim 16, wherein the protein sample is incubated with the amphipathic co-solvent at at least 40° C. for at least 10 minutes prior to the addition of the co-reagent.
